(19) United States
(12) Patent Application Publication
(10) Pub. No.: US 2024/0108800 A1
(43) Pub. Date: Apr. 4, 2024

(54) DUAL WHEEL ACTUATOR

(71) Applicant: INSULET CORPORATION, Acton, MA (US)

(72) Inventors: Soroush KAMRAVA, Everett, MA (US); Steven CARDINALI, Tewksbury, MA (US); Daniel ALLIS, Boxford, MA (US)

(21) Appl. No.: 18/474,682

(22) Filed: Sep. 26, 2023

Related U.S. Application Data
(60) Provisional application No. 63/377,767, filed on Sep. 30, 2022.

(51) Int. Cl. A61M 5/142; A61M 5/145

(52) U.S. Cl. CPC A61M 5/14216; A61M 5/1454; A61M 2005/14506; A61M 2205/0266

(57) ABSTRACT

A drug delivery device drive mechanism including a first ratchet wheel having a first ratchet gear coupled to a first spur gear, a second ratchet wheel having a second ratchet gear coupled to a second spur gear, wherein the first ratchet wheel and the second ratchet wheel are coplanar and gear teeth of the first spur gear mesh with gear teeth the second spur gear, and an actuation mechanism including a pusher interface coupled to an actuator, the pusher interface having at least one pusher tab operable to physically contact the first ratchet gear.

### RELATED APPLICATIONS

This application claims the benefit of U.S. Provisional Patent Application No. 63/377,767, filed Sep. 30, 2022, the entire contents of which are incorporated herein by reference in its entirety.

### TECHNICAL FIELD

The disclosed embodiments generally relate to medication delivery. More particularly, the disclosed embodiments relate to techniques, processes, systems, and devices that use a dual wheel actuator to deliver a medicament to a user.

### BACKGROUND

Wearable drug delivery devices can include a reservoir for storing a liquid drug. A drive mechanism is operated to expel the stored liquid drug from the reservoir for delivery to a user. In some cases, the drive mechanism includes multiple ratchet wheels that provide the necessary angular motion/torque to run the pump. However, based on the arrangement of such ratchet wheels, the drive mechanism may occupy a relatively large volume inside the drug delivery device, increasing the size and cost of the device. In addition, present drive mechanisms utilize a significant portion of the electrical energy stored in the energy storage devices of a wearable drug delivery device.

Accordingly, there is a need for a more power efficient and compact drug delivery device drive mechanism for expelling a liquid drug from a reservoir.

### SUMMARY

At least one aspect of the present disclosure is directed to a drug delivery device drive mechanism as defined in claim 1. The drug delivery device drive mechanism may include a first ratchet wheel having a first ratchet gear coupled to a first spur gear, a second ratchet wheel having a second ratchet gear coupled to a second spur gear, wherein the first ratchet wheel and the second ratchet wheel are coplanar and gear teeth of the first spur gear mesh with gear teeth the second spur gear, and an actuation mechanism including a pusher interface coupled to an actuator, the pusher interface having at least one pusher tab operable to physically contact and incrementally rotate the first ratchet gear or the second ratchet gear, respectively. Alternatively, the pusher interface may physically contact the first spur gear or the second spur gear. In this case, the ratchet gears are not needed.

Another aspect of the present disclosure is directed to a drug delivery device drive mechanism. The drug delivery device drive mechanism includes a first ratchet wheel having a first ratchet gear coupled to a first spur gear, a second ratchet wheel having a second ratchet gear coupled to a second spur gear, wherein gear teeth of the first spur gear mesh with gear teeth of the second spur gear at an angle greater than zero degrees and less than 180 degrees, and an actuation mechanism including a pusher interface coupled to an actuator, the pusher interface having a pair of pusher tabs operable to physically contact the first ratchet gear and the second ratchet gear.

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1 illustrates a schematic diagram of a drug delivery system according to embodiments of the present disclosure;

FIG. 2A illustrates a schematic diagram of a drug delivery system according to embodiments of the present disclosure;

FIG. 2B illustrates a perspective view of an example of a drug delivery system of FIG. 2A according to embodiments of the present disclosure;

FIG. 2C illustrates a perspective view of an example of a drive mechanism of a drug delivery system of FIG. 2A according to embodiments of the present disclosure;

FIG. 2D illustrates a perspective view of the example drive mechanism of FIG. 2C according to embodiments of the present disclosure;

FIG. 3A illustrates a drive mechanism of a liquid drug delivery device according to embodiments of the present disclosure;

FIG. 3B illustrates an actuation mechanism of a drive mechanism of FIG. 3A according to embodiments of the present disclosure;

FIGS. 4A, 4B illustrate an example operation of a drive mechanism of FIG. 3A according to embodiments of the present disclosure;

FIG. 5 illustrates a drive mechanism of a liquid drug delivery device according to embodiments of the present disclosure;

FIG. 6 illustrates a drive mechanism of a liquid drug delivery device according to embodiments of the present disclosure;

FIG. 7 illustrates a drive mechanism of a liquid drug delivery device according to embodiments of the present disclosure;

FIGS. 8A, 8B illustrate an example operation of a drive mechanism of a liquid drug delivery device according to embodiments of the present disclosure;

FIG. 9 illustrates a drive mechanism of a liquid drug delivery device according to embodiments of the present disclosure;

FIG. 10 illustrates a drive mechanism of a liquid drug delivery device according to embodiments of the present disclosure;

FIG. 11 illustrates a drive mechanism of a liquid drug delivery device according to embodiments of the present disclosure;

FIGS. 12A, 12B illustrate an example operation of a drive mechanism of a liquid drug delivery device according to embodiments of the present disclosure; and

FIGS. 13A, 13B illustrate an example operation of a drive mechanism of a liquid drug delivery device according to embodiments of the present disclosure.

The drawings are not necessarily to scale. The drawings are merely representations, not intended to portray specific parameters of the disclosure. The drawings are intended to depict exemplary embodiments of the disclosure, and therefore are not to be considered as limiting in scope. Furthermore, certain elements in some of the figures may be omitted, or illustrated not-to-scale, for illustrative clarity. Still furthermore, for clarity, some reference numbers may be omitted in certain drawings.

### DETAILED DESCRIPTION

Systems, devices, and methods in accordance with the present disclosure will now be described more fully hereinafter with reference to the accompanying drawings, where one or more embodiments are shown. The systems, devices, and methods may be embodied in many different forms and are not to be construed as being limited to the embodiments set forth herein. Instead, these embodiments are provided so the disclosure will be thorough and complete, and will fully convey the scope of methods and devices to those skilled in the art. Each of the systems, devices, and methods disclosed herein provides one or more advantages over conventional systems, components, and methods.

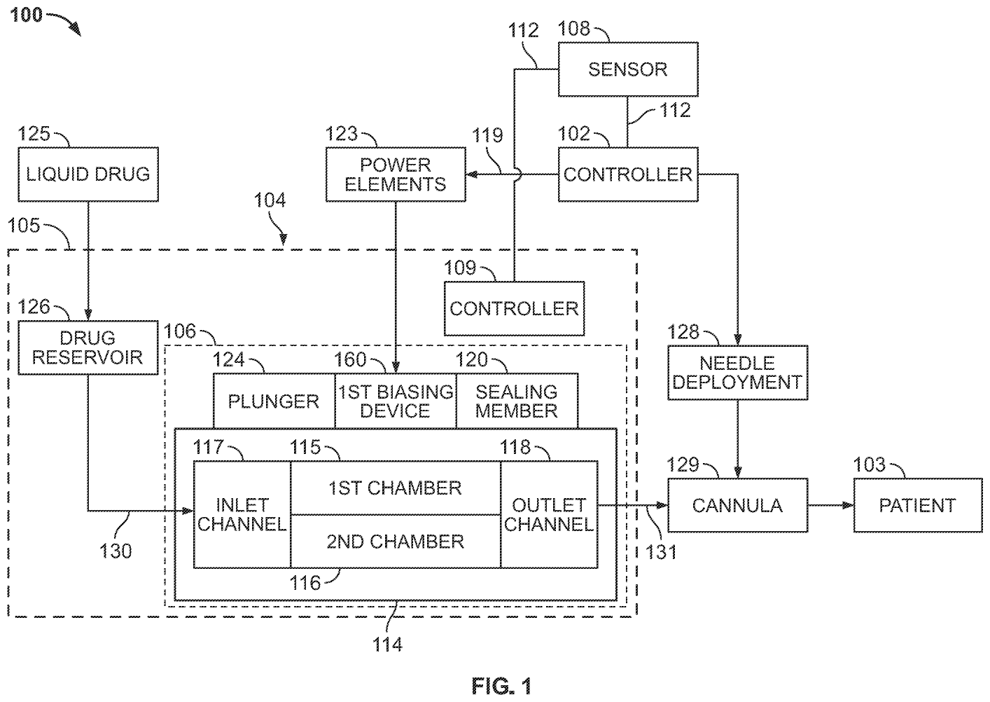

FIG. 1 illustrates a simplified block diagram of an example system 100. The system 100 may be a wearable or on-body drug delivery device and/or an analyte sensor attached to the skin of a patient 103. The system 100 may include a controller 102, a pump mechanism 104 (hereinafter “pump 104”), and a sensor 108. The sensor 108 may be one or more of a glucose or other analyte monitor such as, for example, a continuous glucose monitor, a ketone sensor, a heart rate monitor, a blood oxygen sensor element, or the like, and may be incorporated into the wearable device. The sensor(s) 108 may, for example, be operable to measure blood glucose (BG) values of a user to generate a measured BG level signal 112. The controller 102, the pump 104, and the sensor(s) 108 may be communicatively coupled to one another via a wired or wireless communication path. For example, each of the controller 102, the pump 104 and the sensor(s) 108 may be equipped with a wireless radio frequency transceiver operable to communicate via one or more communication protocols, such as Bluetooth®, or the like. As will be described in greater detail herein, the system 100 may also include a pump 104 which includes a drive mechanism 106 having at least one housing 114 defining a pump chamber 115, a channel chamber 116, an inlet channel 117, and an outlet channel 118. The drive mechanism 106 may further include a resilient sealing member 120 enclosing the pump chamber 115, and a first biasing device 160 (e.g., shape memory alloy (SMA) wire or the like) operable to bias or move a plunger 124 relative to the pump chamber 115, as will be described in greater detail herein. In an example, the drive mechanism 106, the controller 109 and the drug reservoir 126 may be incorporated in a housing 105, which may be one or more parts that may be a combination of reusable and disposable parts. The housing 105 may be formed from one or more materials, such as a plastic, a metal or the like. The system 100 may include additional components not shown or described for the sake of brevity.

The controller 102 may receive a desired BG level signal, which may be a first signal, indicating a desired BG level or range for the patient 103. The desired BG level or range may be stored in memory of a controller 109 on pump 104, received from a user interface of the controller 102, or another device, or by an algorithm within controller 109 (or controller 102). The sensor(s) 108 may be coupled to the patient 103 and operable to measure an approximate value of a BG level of the user. In response to the measured BG level or value, the sensor(s) 108 may generate a signal indicating the measured BG value. As shown in the example, the controller 102 may also receive from the sensor(s) 108 via a communication path, the measured BG level signal 112, which may be a second signal.

Based on the desired BG level and the measured BG level signal 112, the controller 102 or controller 109 may generate one or more control signals for directing operation of the pump 104. For example, one control signal 119 from the controller 102 or controller 109 may cause the pump 104 to turn on, or activate one or more power elements 123 operably connected with the pump 104. In the case where the first biasing device 160 is an SMA wire, activation of the SMA wire by the power element 123 may cause the SMA wire to change shape and/or length, which in turn may cause movement of the plunger 124 and the resilient sealing member 120. The specified amount of a liquid drug 125 (e.g., insulin, GLP-1, pramlintide, or a co-formulation of insulin, GLP-1 or pramlintide; a chemotherapy drug; a blood thinner; a pain medication; an arthritis drug; or the like) may be drawn from the reservoir 126 into the pump chamber 115, through the inlet channel 117, in response to a change in pressure due to the change in configuration of the resilient sealing member 120 and the plunger 124. In some examples, the specified amount of the liquid drug 125 to be delivered may be determined based on a difference between the desired BG level and the actual BG level signal 112. For example, specified amount of the liquid drug 125 may be determined as an appropriate amount of insulin to drive the measured BG level of the user toward the desired BG level. Based on operation of the pump 104, as determined by the control signal 119, the patient 103 may receive the liquid drug from a reservoir 126. The system 100 may operate as a closed-loop system, an open-loop system, or as a hybrid system. In an exemplary closed-loop system, the controller 109 may direct operation of the pump 104 without input from the user or controller 102, and may receive BG level signal 112 from the sensor(s) 108. The sensor(s) 108 may be housed within the pump 104 or may be housed in a separate device and communicate wirelessly directly with the pump 104 (e.g., with controller 109) or with an external controller 102.

As further shown, the system 100 may include a needle deployment component 128 in communication with the controller 102 or the controller 109. Though shown separately, needle deployment component 128 may be integrated within pump 104. The needle deployment component 128 may include a needle and/or cannula 129 deployable into the patient 103 and may have one or more lumens and one or more holes at a distal end thereof. The cannula 129 may form a portion of a fluid path coupling the patient 103 to the reservoir 126. More specifically, the inlet channel 117 may be coupled to the reservoir 126 by a first fluid path component 130. The first fluid path component 130 may be of any size and shape and may be made from any material. The first fluid path component 130 enables fluid, such as the liquid drug 125 in the reservoir 126, to be transferred to the drive mechanism 106.

As further shown, the outlet channel 118 may be coupled to the cannula 129 by a second fluid path component 131. The second fluid path component 131 may be of any size and shape and may be made from any material. The second fluid path component 131 may be connected to the cannula 129 to allow fluid expelled from the pump 104 to be provided to the patient 103. The first and second fluid path components 130 and 131 may be rigid, flexible, or a combination thereof.

The controller 102/109 may be implemented in hardware, software, or any combination thereof. The controller 102/109 may, for example, be a processor, a logic circuit or a microcontroller coupled to a memory. The controller 102/109 may maintain a date and time as well as provide other functions (e.g., calculations or the like) performed by processors. The controller 102/109 may be operable to execute an artificial pancreas (AP) algorithm stored in memory (not shown in this example) that enables the controller 102/109 to direct operation of the pump 104. For example, the controller 102/109 may be operable to receive an input from the sensor(s) 108, wherein the input comprises analyte level data, such as blood glucose data or levels over time. Based on the analyte level data, the controller 102/109 may modify the behavior of the pump 104 and resulting amount of the liquid drug 125 to be delivered to the patient 103.

The power elements 123 may be a battery, a supercapacitor, a piezoelectric device, or the like, for supplying electrical power to the pump 104. In other embodiments, the power element 123, or an additional power source (not shown), may also supply power to other components of the pump 104, such as the controller 102, memory, the sensor(s) 108, and/or the needle deployment component 128.

In an example, the sensor(s) 108 may be a device communicatively coupled to the controller 102 and may be operable to measure a blood glucose value at a predetermined time interval, such as approximately every 5 minutes, 1 minute, or the like. The sensor(s) 108 may provide a number of blood glucose measurement values to the AP application.

In some embodiments, the pump 104, when operating in a normal mode of operation, provides insulin stored in the reservoir 126 to the patient 103 based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like) provided by the sensor(s) 108 or other functional elements of the system 100 or pump 104. For example, the pump 104 may contain analog and/or digital circuitry that may be implemented at the controller 102/109 for controlling the delivery of the drug or therapeutic agent. The circuitry used to implement the controller 102/109 may include discrete, specialized logic and/or components, an application-specific integrated circuit, a microcontroller or processor that executes software instructions, firmware, programming instructions or programming code enabling, for example, an AP application stored in memory, or any combination thereof. For example, the controller 102/109 may execute a control algorithm and other programming code that may make the controller 102/109 operable to cause the pump to deliver doses of the drug or therapeutic agent to a user at predetermined intervals or as needed to bring blood glucose measurement values to a target blood glucose value. The size and/or timing of some of the doses may be pre-programmed, for example, into the AP application by the patient 103 or by a third party (such as a health care provider, a parent or guardian, a manufacturer of the wearable drug delivery device, or the like) using a wired or wireless link, or may be calculated iteratively by the controller 102 or controller 109, such as every 5 minutes.

Although not shown, in some embodiments, the sensor(s) 108 may include a processor, memory, a sensing or measuring device, and a communication device. The memory of the sensor(s) 108 may store an instance of an AP application as well as other programming code and be operable to store data related to the AP application.

In various embodiments, the processor of the sensor(s) 108 may include discrete, specialized logic and/or components, an application-specific integrated circuit, a microcontroller or processor that executes software instructions, firmware, programming instructions stored in memory, or any combination thereof.

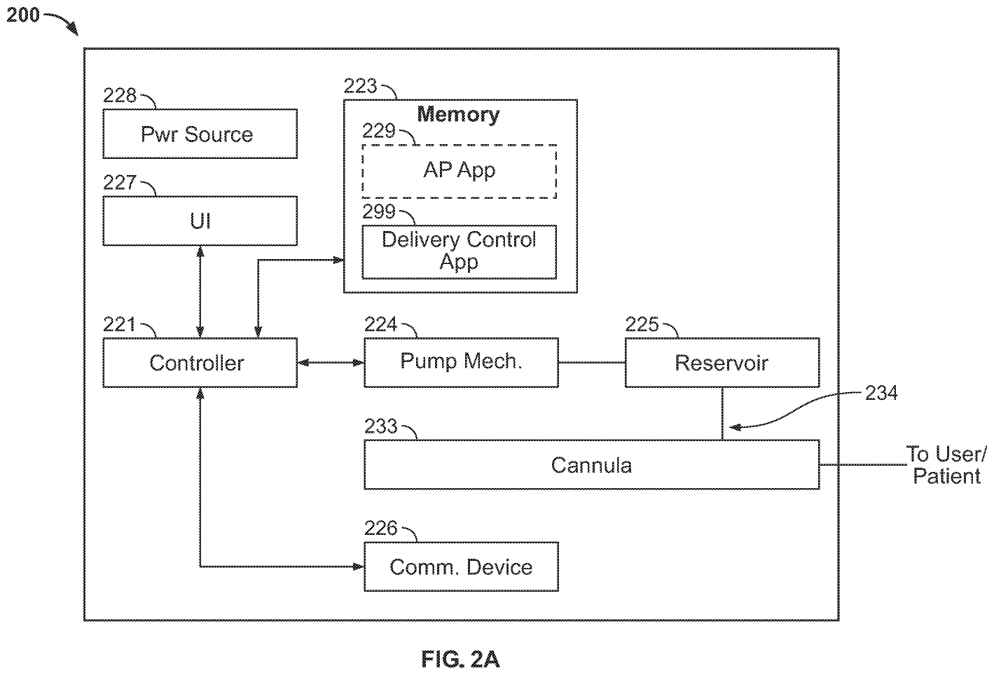

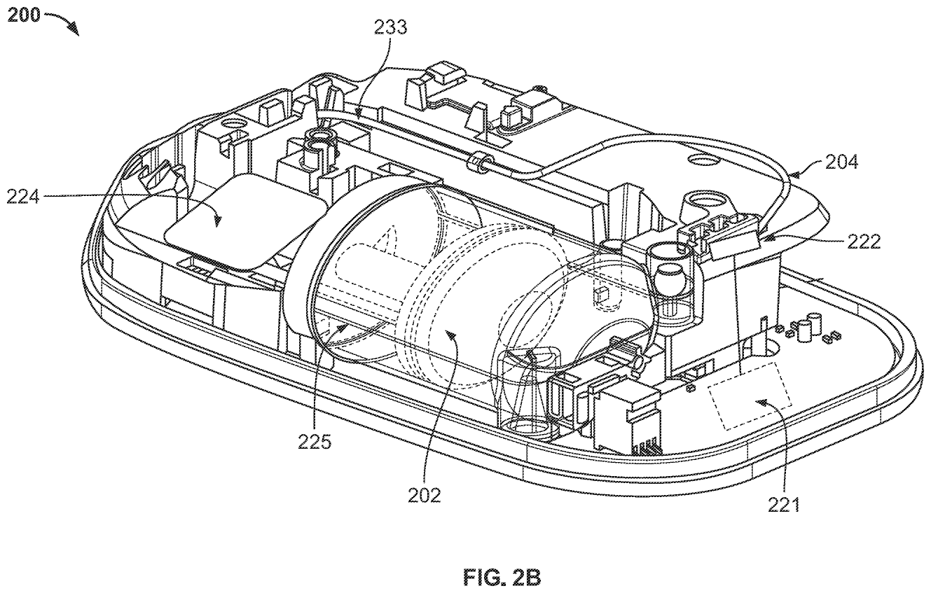

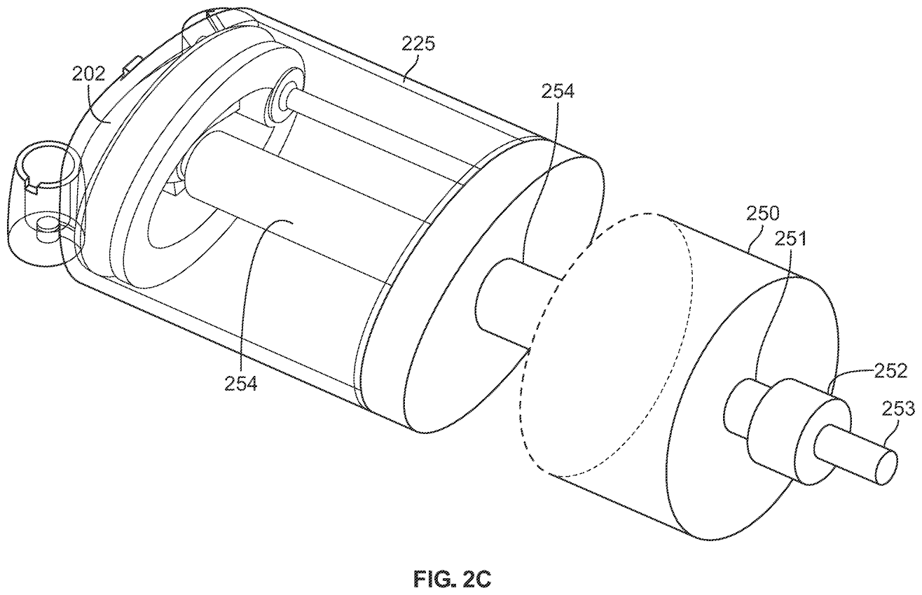

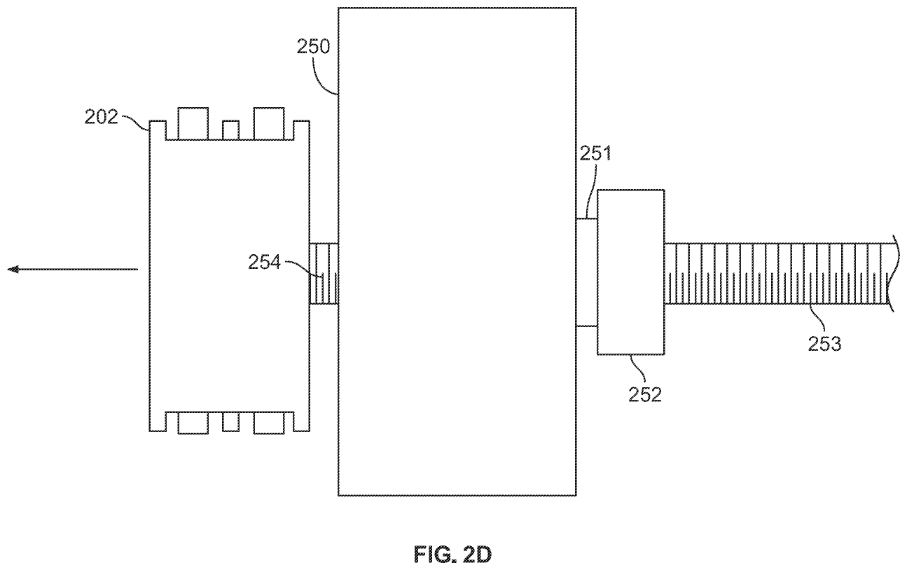

FIG. 2A illustrates a simplified block diagram of another example system 200. The system 200 may include a controller 221, a memory 223, an AP application 229 and delivery control application 299 stored in the memory 223, a pump mechanism 224, a communication device 226, user interface 227, and a power source 228. The memory 223 may be operable to store programming code and applications including a delivery control application 299, the AP application 229 and data. The delivery control application 299 and the AP application 229 may optionally be stored on other devices.

The controller 221 may be coupled to the pump mechanism 224 and the memory 223. The controller 221 may include logic circuits, a clock, a counter or timer as well as other processing circuitry, and be operable to execute programming code and the applications stored in the memory 223 including the delivery control application 299. A communication device 226 may be communicatively coupled to the controller 221 and may be operable to wirelessly communicate with an external device, such as a personal diabetes management device, a smart device such as a smartphone and/or a smartwatch, or the like.

The pump mechanism 224 may be operable to deliver a drug, like insulin, at a fixed or variable rate. For example, an AP application or AID algorithm executing on a personal diabetes management device or a smart phone may determine or be informed that a user's total daily insulin (e.g., bolus and basal deliveries) is 48 units per 24 hours, which may translate to an exemplary physiological basal dosage rate of 1 unit per hour (48/24/2 (assuming a 1:1 basal/bolus ratio)) that may be determined according to a diabetes treatment plan. Of course, the pump mechanism 224 may be operable to deliver insulin at rates different from the example physiological dosage rate of 1 unit per hour. In an example, the system 200 may be attached to the body of a user, such as a patient or diabetic via, for example, by an adhesive, (e.g., directly attached to the skin of the user) and may deliver any therapeutic agent, including any drug or medicine, such as insulin, morphine, or the like, to the user. In an example, a surface of the system 200 may include an adhesive (not shown) to facilitate attachment to a user. The system 200 may, for example, be worn on a belt or in a pocket of the user and the liquid drug may be delivered to the user via tubing to an infusion site on the user.

In various examples, the system 200 may be an automatic, wearable drug delivery device. For example, the system 200 may include a reservoir 225 configured to hold a liquid drug (such as insulin), a needle and/or cannula 233 for delivering the drug into the body of the user (which may be done subcutaneously, intraperitoneally, or intravenously), and a pump mechanism 224, or other drive mechanism, for transferring the drug from the reservoir 225, through a needle or cannula 233, and into the user.

The pump mechanism 224 may be fluidly coupled to reservoir 225, and communicatively coupled to the medical device controller 221. The pump mechanism 224 may be coupled to the reservoir 225 and operable to output the liquid drug from the reservoir 225 via a fluid delivery path and out of the cannula 233. The pump mechanism 224 may have mechanical parameters and specifications, such as a pump resolution, that indicate mechanical capabilities of the pump mechanism. The pump mechanism 224 may also have electrical connections to control circuitry (not shown) that is operable to control operation of the pump mechanism 224. The pump resolution is a fixed amount of insulin the pump mechanism 224 delivers in a pump mechanism pulse, which is an actuation of the pump mechanism for a preset time period. Actuation may be when power from the power source 228 is applied to the control circuitry coupled to the pump mechanism 224 and the pump mechanism 224 operates to pump a fixed amount of insulin in a preset amount of time from the reservoir 225. Alternatively, the pump mechanism 224 may be substantially mechanical in structure and operation and utilize mechanical energy storage devices, such as springs or other biasing members to operate the pump mechanism 224.

The cannula 233 of FIG. 2A may be coupled to the reservoir 225 via a fluid delivery path 234. The cannula 233 may be operable to output the liquid drug to a user when the cannula 233 is inserted in the user.

The system 200 may also include a power source 228, such as a battery, a supercapacitor, a piezoelectric device, or the like, that is operable to supply electrical power to the pump mechanism 224 and/or other components (such as the controller 221, memory 223, and the communication device 226) of the system 200.

As shown in FIG. 2B, the system 200 may include a plunger 202 positioned within the reservoir 225. An end portion or stem of the plunger 202 can extend outside of the reservoir 225. The pump mechanism 224 may, under control of the controller 221, be operable to cause the plunger 202 to expel the fluid, such as a liquid drug (not shown) from the reservoir 225 and into a fluid component 204 and cannula 233 by advancing into the reservoir 225. In various examples, a pressure sensor, such as that shown at 222, may be integrated anywhere along the overall fluid delivery path of the system 200, which includes the reservoir 225, the fluid delivery path component 204, and the cannula 233.

The controller 221 may be implemented in hardware, software, or any combination thereof. In various examples, the controller 221 can be implemented as dedicated hardware (e.g., as an application specific integrated circuit (ASIC)). The controller 221 may be a constituent part of the system 200, can be implemented in software as a computational model, or can be implemented external to the system 200 (e.g., remotely). The controller 221 may be configured to communicate with one or more sensors (e.g., sensor(s) 108 of FIG. 1).

As described above, a reservoir, such as 225, may be included in a drug delivery device to store a liquid drug (e.g., insulin). For example, the reservoir 225 may be filled, or partially filled, with a liquid drug or a liquid drug solution. In one example, a liquid drug solution is a mixture of the liquid drug and added preservatives. The reservoir may store the liquid drug until all of the liquid drug has been dispensed (e.g., into a patient via a cannula). As such, the liquid drug (or solution) may remain in the reservoir for a period of time (e.g., 1 day, 3 days, 1 week, 2 weeks, etc.).

FIG. 2C illustrates an example of a reservoir coupled to a drive mechanism 250 that may be included in the pump mechanism 224. Likewise, FIG. 2D illustrates a perspective view of the drive mechanism 250. As disclosed in later examples, the drive mechanism 250 (shown in more detail in later examples) may include a drive wheel, co-planar ratchet wheels, and an actuator. In one example, the set of ratchet wheels are attached to the drive wheel. The drive wheel may be coupled to a plunger 202 via an elongated shaft 254. At a high level, the ratchet wheels are engaged by the actuator to incrementally drive the drive wheel and advance the plunger 202 and the elongated shaft 254 into the reservoir 225. The elongated shaft 245 advances the plunger 202 to dispense the liquid drug out of the reservoir 225. In one example, a pump mechanism coupling 251 is operable to connect either the first ratchet wheel or the second ratchet wheel of the drive mechanism 250 to a drive element 252. The drive element 252 includes (or is otherwise coupled to) a lead screw 253 that is coupled to the plunger 202 (e.g., via the elongated shaft 254). The pump mechanism 224 is operable to rotate the lead screw 253 (e.g., via the drive mechanism 250) and move the plunger 202 to expel the liquid drug from the reservoir 225. In some examples, the rotation of the lead screw 253 causes the elongated shaft 254 to advance within the reservoir 225, for example by providing a fixed nut assembly. Alternatively, a rotation of the lead screw may be prevented (for example by providing a fixed connection between the lead screw and the plunger, wherein the plunger and the reservoir have a non-circular—e.g. oval—cross-sectional shape which prevents a rotation of the plunger within the reservoir). In this case, an internal threading of one of the ratchet wheels may engage the external threading of the lead screw such that a rotation of the ratchet wheel results in a linear movement of the lead screw.

Improved drive mechanisms for liquid drug delivery devices are provided herein. In at least one embodiment, the drive mechanism is a dual wheel actuator with coplanar ratchet wheels. In one example, a first ratchet wheel includes a first spur gear having gear teeth that mesh with gear teeth of a second spur gear of a second ratchet wheel. In some examples, the co-planar arrangement of the first and second ratchet wheels allows the drive mechanism to have a compact form factor than a co-axial arrangement provided in other implementations. In this context, a co-axial arrangement refers to an arrangement where the first and second rachet wheels rotate about the same axis.

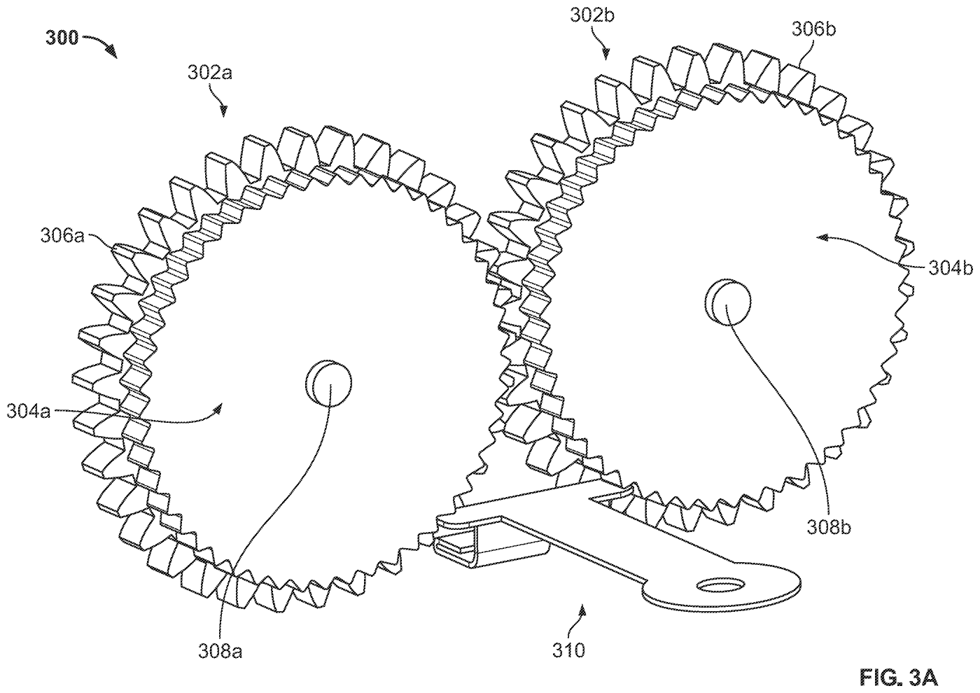

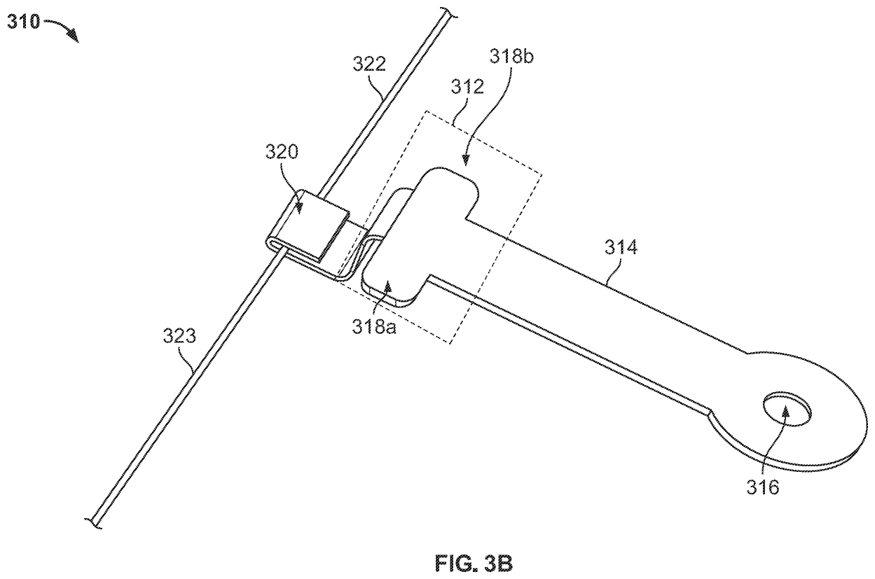

FIG. 3A illustrates a drive mechanism 300 of a liquid drug delivery device in accordance with aspects described herein. In one example, the drive mechanism 300 is operable for use with the systems 100, 200 of FIGS. 1 and 2A-2B. For example, the drive mechanism 300 may be included in the pump mechanism 224 of the system 200.

The drive mechanism 300 includes a first ratchet wheel 302a and a second ratchet wheel 302b. As shown, the first ratchet wheel 302a and the second ratchet wheel 302b are coplanar. The first ratchet wheel 302a includes a first ratchet gear 304a and a first spur gear 306a. The first ratchet gear 304a may be coupled to the first spur gear 306a (e.g., via a first shaft 308a). The first ratchet gear 304a includes a first plurality of ratchet gear teeth and the first spur gear 304a includes a first plurality of spur gear teeth. The first plurality of ratchet gear teeth and the first plurality of spur gear teeth may have different spacings and/or sizes. Likewise, the second ratchet wheel 302b includes a second ratchet gear 304b and a second spur gear 306b. The second ratchet gear 304b may be coupled to the second spur gear 306b (e.g., via a second shaft 308b). The second ratchet gear 304b includes a second plurality of ratchet gear teeth and the second spur gear 306b includes a second plurality of spur gear teeth. The second plurality of ratchet gear teeth and the second plurality of spur gear teeth may have different spacings and/or sizes. In one example, the first plurality of spur gear teeth of the first spur gear 306a mesh with the second plurality of spur gear teeth of the second spur gear 306b.

The drive mechanism 300 includes an actuation mechanism 310. In one example, the actuation mechanism 310 is configured to engage with the first plurality of ratchet gear teeth of the first ratchet gear 304a and the second plurality of ratchet gear teeth of the second ratchet gear 304b. In all embodiments of the present disclosure, the actuation mechanism may e.g. be a linear actuation mechanism or a rotatable actuation mechanism. As shown in FIG. 3B, the actuation mechanism 310 includes a pusher interface 312, an actuator arm 314, and a pivot point 316. The pusher interface 312 is coupled to a first end of the actuator arm 314 and the pivot point 316 is coupled to a second, opposite end of the actuator arm 314. The pusher interface 312 includes a first pusher tab 318a and a second pusher tab 318b. In one example, the first pusher tab 318a is operable to physically contact the first plurality of ratchet gear teeth of the first ratchet gear 304a and the second pusher tab 318b is operable to physically contact the second plurality of ratchet gear teeth of the second ratchet gear 304b. In some examples, the actuation mechanism 310 is positioned such that the pusher interface 312 is substantially perpendicular to the first ratchet wheel 302a. In other words, the pusher interface 312 may be substantially perpendicular to a gear face of the first ratchet wheel 302a (or the first ratchet gear 304a).

In some examples, the pusher interface 312 is coupled to a crimp connection 320. In other examples, the crimp connection 320 can be coupled to the actuator arm 314. The crimp connection 320 is coupled to at least one actuator (not shown) via biasing members 322, 323. In one example, biasing members 322, 323 correspond to SMA wires. In another example, biasing member 322 corresponds to a SMA wire and biasing member 323 corresponds to a spring. More specifically, a SMA wire may be positioned on one side of crimp connection 320 and connected thereto, to bias actuator arm 314 in one direction (upon activation of the SMA wire), and a spring may be positioned on the other side of crimp connection 320 and connected thereto, to bias actuator 314 in an opposite direction (upon deactivation of the SMA wire). The at least one actuator may alternate between causing the first pusher tab 318a to contact the first plurality of ratchet gear teeth of the first ratchet gear 304a and causing the second pusher tab 318b to contact the second plurality of ratchet gear teeth of the second ratchet gear 304b. For example, the at least one actuator may pull the crimp connection 320 via biasing member 323 causing the actuator arm 314 to rotate about the pivot point 316 such that the first pusher tab 318a contacts a gear tooth of the first plurality of ratchet gear teeth. The first pusher tab 318a may be oriented to be perpendicular to a respective gear tooth (e.g., the gear tooth being contacted) of the plurality of ratchet gear teeth of the first ratchet gear 304a. Likewise, the at least one actuator may pull the crimp connection 320 via biasing member 322 causing the actuator arm 314 to rotate about the pivot point 316 such that the second pusher tab 318b contacts a gear tooth of the second plurality of ratchet gear teeth. The second pusher tab 318b may be oriented to be perpendicular to a respective gear tooth (e.g., the gear tooth being contacted) of the plurality of ratchet gear teeth of the second ratchet gear 304b.

In one example, by adjusting the distance between the pusher interface 312 (or the pusher tabs 318a, 318b) and the pivot point 316, the stroke length and/or force of the biasing members 322, 323 can be tuned to reach to a proper balance for driving the ratchet wheels 302a, 302b. In other examples, the distance between the crimp connection 320 and the pivot point 316 may be adjusted to achieve the proper balance.

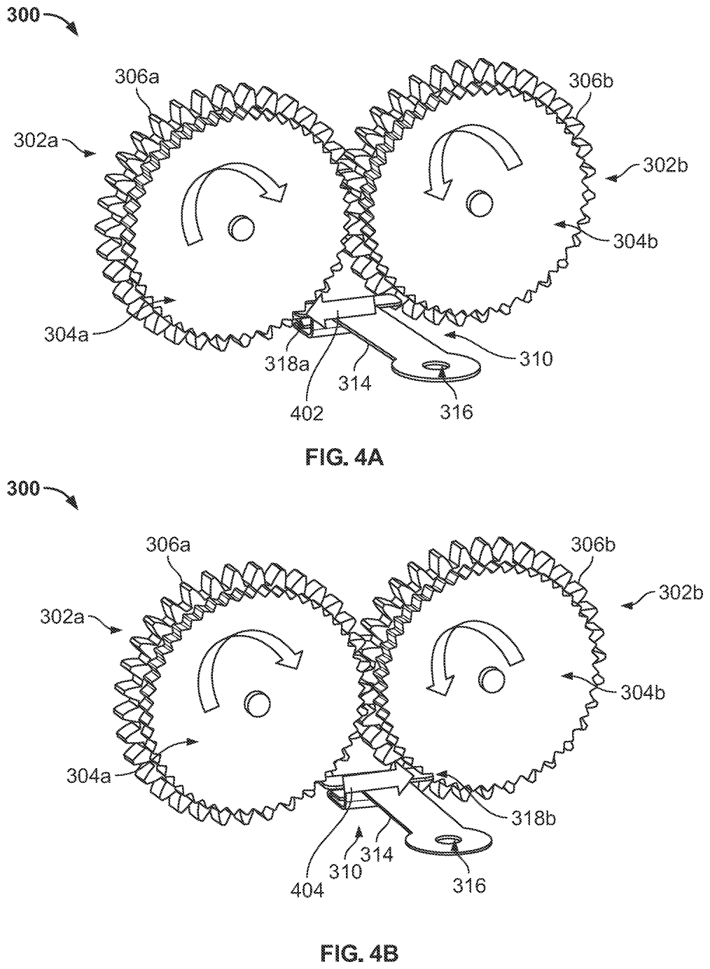

FIGS. 4A and 4B illustrate an example operation of the drive mechanism 300. In one example, FIG. 4A represents a first operational state of the drive mechanism 300 and FIG. 4B represents a second operational state of the drive mechanism 300.

As shown in FIG. 4A, in the first operational state, the actuation mechanism 310 is pulled (e.g., via the crimp connection 320) in a first linear direction 402. The actuator arm 314 rotates about the pivot point 316 causing the first pusher tab 318a to contact a respective gear tooth of the first plurality of ratchet gear teeth of the first ratchet gear 304a. The force applied to the respective gear tooth by the first pusher tab 318a causes the first ratchet gear 304a to rotate in a first rotational direction (e.g., clockwise). Being that the first ratchet gear 304a is coupled to the first spur gear 306a, the rotation of the first ratchet gear 304a causes the first spur gear 306a to rotate in the first rotational direction. In one example, the first ratchet wheel 302a (e.g., the first ratchet gear 304a and the first spur gear 306a) rotates about a first axis in the first rotational direction. In some examples, the first axis is parallel to the first shaft 308a.

The plurality of spur gear teeth of the first spur gear 306a apply a force to the plurality of spur gear teeth of the second spur gear 306b that causes the second spur gear 306b to rotate in a second rotational direction (e.g., counter-clockwise). Being that the second ratchet gear 304b is coupled to the second spur gear 306b, the rotation of the second spur gear 306b causes the second ratchet gear 304b to rotate in the second rotational direction. In one example, the second ratchet wheel 302b (e.g., the second ratchet gear 304b and the second spur gear 306b) rotates about a second axis in the second rotational direction. In some examples, the second axis is parallel to the second shaft 308b. The second axis may be parallel to the first axis. It should be appreciated that the second pusher tab 318b will be disengaged from the second ratchet gear 304b when the first pusher tab 318a is in contact with the first ratchet gear 304a (e.g., in the first operational state).

As shown in FIG. 4B, in the second operational state, the actuation mechanism 310 is pulled (e.g., via the crimp connection 320) in a second linear direction 404. In some examples, the first and second linear directions 402, 404 are opposite directions (e.g., left and right). The actuator arm 314 rotates about the pivot point 316 causing the second pusher tab 318b to contact a respective gear tooth of the second plurality of ratchet gear teeth of the second ratchet gear 304b. The force applied to the respective gear tooth by the second pusher tab 318b causes the second ratchet gear 304b to rotate about the second axis in the second rotational direction. Being that the second ratchet gear 304b is coupled to the second spur gear 306b, the rotation of the second ratchet gear 304b causes the second spur gear 306b to rotate about the second axis in the second rotational direction. The plurality of spur gear teeth of the second spur gear 306b apply a force to the plurality of spur gear teeth of the first spur gear 306a that causes the first spur gear 306a to rotate about the first axis in the first rotational direction. Being that the first ratchet gear 304a is coupled to the first spur gear 306a, the rotation of the first spur gear 306a causes the first ratchet gear 304a to rotate about the first axis in the first rotational direction. It should be appreciated that the first pusher tab 318a will be disengaged from the first ratchet gear 304a when the second pusher tab 318b is in contact with the second ratchet gear 304b (e.g., in the second operational state).

The drive mechanism 300 is operated to convert the linear motion of the biasing members 322, 323 into the rotational motion of the ratchet wheels 302a, 302b. Alternating the drive mechanism 300 between the first and second operational states results in an incremental rotation of each ratchet wheel 302a, 302b. As such, by coupling one of the ratchet wheels 302a, 302b to a pumping mechanism (e.g., pump mechanism 224), the incremental rotational motion can be used to actuate the pump and deliver a liquid drug to a patient. For example, a pump mechanism coupling may be operable to connect either the first ratchet wheel 302a or the second ratchet wheel 302b to a drive element (e.g., the drive wheel 256 of FIG. 2C). The drive element may include a lead screw (and optionally a tube nut over the lead screw) coupled to a plunger (e.g., the plunger 202 of FIG. 2C). The drive mechanism 300 is operable to rotate the lead screw via the drive element and move the plunger to expel a liquid drug from a reservoir, pump chamber, or the like (e.g., the reservoir 225 of FIG. 2C). In some examples, the size (e.g., diameter) of the ratchet wheels 302a, 302b controls the dosage provided by the pump mechanism. For example, larger ratchet wheels 302a, 302b may enable the drive mechanism 300 to operate the pump mechanism with a higher dosage resolution (i.e., smaller dosage sizes per pulse of the pump mechanism, which may be defined as one actuation of actuator arm 314).

As described above, the actuation mechanism 310 is actively pulled in multiple linear directions using at least one actuator and at least one biasing member 322, 323. For example, the crimp connection 320 of the actuation mechanism 310 is actively pulled via biasing member 323 in the first linear direction 402 and via biasing member 322 in the second linear direction 404. In other examples, two different types of biasing members may be used such that the actuation mechanism 310 is actively pulled in one linear direction (e.g., with an SMA wire) and passively pulled in the other linear direction (e.g., with a return spring).

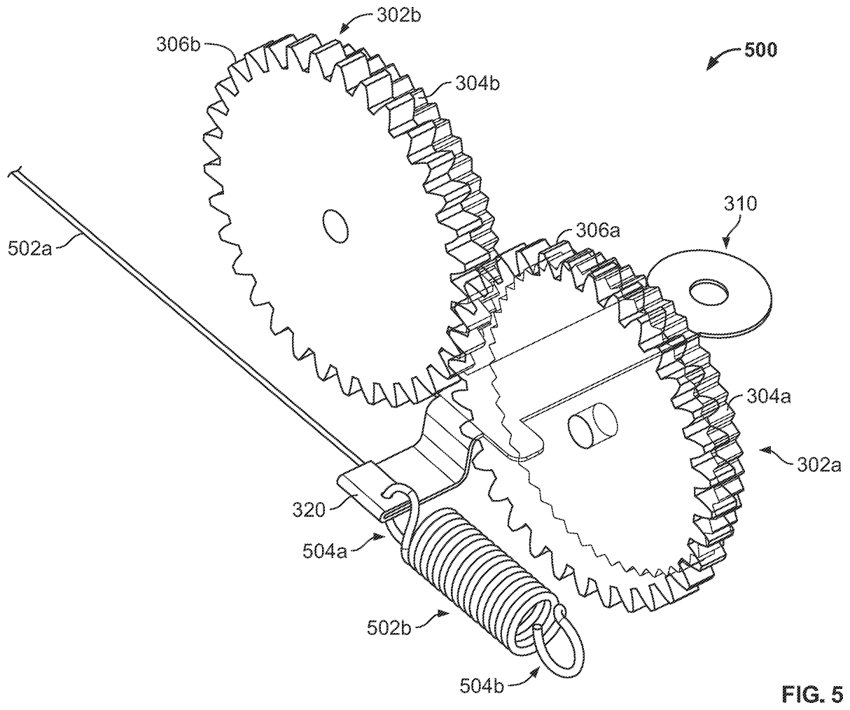

FIG. 5 illustrates a drive mechanism 500 in accordance with aspects described herein. In one example, the drive mechanism 500 is substantially the same as the drive mechanism 300 of FIGS. 3A-4B, except the drive mechanism 500 includes a first biasing member 502a shown as a wire (e.g., a SMA wire) and a second biasing member 502b shown as a spring. In some examples, the first biasing member 502a is an SMA wire and the second biasing member 502b is a return spring. As shown, a first end 504a of the second biasing member 502b is coupled to the crimp connection 320 of the actuation mechanism 310. A second end 504b of the second biasing member 502b may be coupled to a fixed point (e.g., an anchor point within the housing of the liquid drug delivery device).

In a first operational state, the actuation mechanism 310 is pulled via the crimp connection 320 in a first linear direction by the first biasing member 502a, causing the actuation mechanism 310 (e.g., the second pusher tab 318b) to contact a respective gear tooth of the second plurality of ratchet gear teeth of the second ratchet gear 304b. The force applied to the respective gear tooth by the actuation mechanism 310 causes the second ratchet wheel 302b (e.g., the second ratchet gear 304b and the second spur gear 306b) to rotate in the first rotational direction. The first end 504a of the second biasing member 502b is pulled in the first linear direction with the crimp connection 320 to extend the second biasing member 502b. As such, in a second operational state, the first biasing member 502a may be released (or relaxed), allowing the actuation mechanism 310 to be pulled via the crimp connection 320 in a second linear direction by the second biasing member 502b. The actuation mechanism 310 (e.g., the first pusher tab 318a) contacts a respective gear tooth of the first plurality of ratchet gear teeth of the first ratchet gear 304a and the force applied to the respective gear tooth by the actuation mechanism 310 causes the first ratchet wheel 302a (e.g., the first ratchet gear 304a and the first spur gear 306a) to rotate in a second rotational direction. The first end 504a of the second biasing member 502b is pulled in the second linear direction with the crimp connection 320 to return the second biasing member 502b to a resting state.

The drive mechanism 500 is operated to convert the linear motion of the biasing members 502a, 502b into the rotational motion of the ratchet wheels 302a, 302b. Alternating the drive mechanism 500 between the first and second operational states results in an incremental rotation of each ratchet wheel 302a, 302b. As such, by coupling one of the ratchet wheels 302a, 302b to a pumping mechanism (e.g., pump mechanism 224), the incremental rotational motion can be used to actuate the pump and deliver a liquid drug to a patient. In some examples, due to the passive configuration of the second biasing member 502b, the drive mechanism 500 may operate with reduced power consumption relative to other drive mechanism configurations (e.g., drive mechanisms that consume power for each biasing member).

As described above, the drive mechanisms 300, 500 each include a ratchet wheel that is configured to be coupled (or connected) to a pumping mechanism to provide a mechanical displacement used for liquid drug delivery.

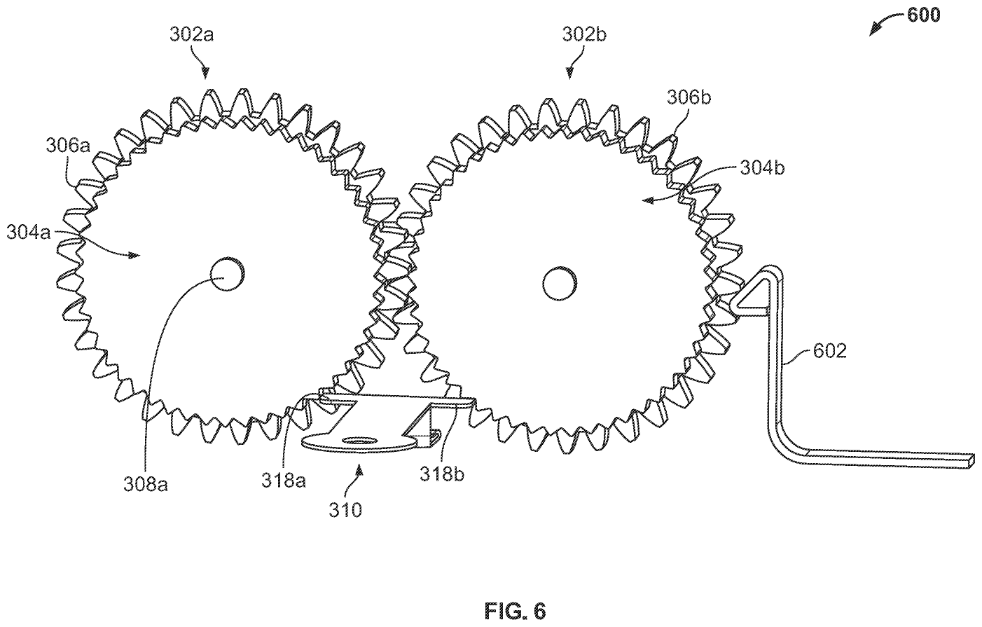

FIG. 6 illustrates a drive mechanism 600 of a liquid drug delivery device in accordance with aspects described herein. In some instances, one ratchet wheel may rotate slightly due to a backlash between the first spur gear and the second spur gear. In one example, the drive mechanism 600 is substantially the same as the drive mechanism 300 of FIGS. 3A-4B, except the drive mechanism 600 includes a pawl mechanism 602. In the illustrated example, the first ratchet wheel 302a is coupled to a pumping mechanism (e.g., via the first shaft 308a) and the incremental rotation of the first ratchet wheel 302a is used to actuate the pump to deliver a liquid drug to a patient. As such, the pawl mechanism 602 is positioned to engage with the second ratchet wheel 302b.

The pawl mechanism 602 may, for example, include a deformable cantilever beam having a sharp bend. In one example, the sharp bend is configured to rest between two gear teeth of the second plurality of spur gear teeth of the second spur gear 306b. The pawl mechanism 602 provides a stabilizing force that prevents free rotation (e.g., backlash) of the second ratchet wheel 302b. Given that the second plurality of spur gear teeth of the second spur gear 306b mesh with the first plurality of spur gear teeth of the first spur gear 306a, a residual force may be applied from the second spur gear 306b to the first spur gear 306a to prevent free rotation of the first ratchet wheel 302a. The pawl mechanism 602 is tuned such that the stabilizing force is less than the force provided by the actuation mechanism 310. For example, the stabilizing force of the pawl mechanism 602 is overcome when the second pusher tab 318b contacts a respective gear tooth of the second plurality of ratchet gear teeth of the second ratchet gear 304b, allowing the second spur gear 306b to rotate with the second ratchet gear 304b. Likewise, the stabilizing force is overcome when the first pusher tab 318a contacts a respective gear tooth of the first plurality of ratchet gear teeth of the first ratchet gear 304a, causing the plurality of spur gear teeth of the first spur gear 306a to apply a force to the plurality of spur gear teeth of the second spur gear 306b to rotate the second spur gear 306b.

As described, the pawl mechanism 602 may be operable to engage with the second plurality of spur gear teeth of the second spur gear 306b. However, it should be appreciated that the pawl mechanism 602 can be configured differently. For example, the pawl mechanism 602 may be operable to engage with the second plurality of ratchet gear teeth of the second ratchet gear 304b. In other examples, the pawl mechanism 602 can be operable to engage with the first plurality of spur gear teeth of the first spur gear 306a or the first plurality of ratchet gear teeth of the first ratchet gear 304a. In some examples, multiple pawl mechanisms can be used. For example, a first pawl mechanism may be operable to engage with the first spur gear 306a or the first ratchet gear 304a and a second pawl mechanism may be operable to engage with the second spur gear 306b or the second ratchet gear 304b. Likewise, a first pawl mechanism may be operable to engage with the first spur gear 306a and a second pawl mechanism may be operable to engage with the first ratchet gear 304a. Similarly, a first pawl mechanism may be operable to engage with the second spur gear 306b and a second pawl mechanism may be operable to engage with the second ratchet gear 304b.

In some examples, a gear train can be used to adjust the mechanical advantage provided by the drive mechanism. For example, rather than driving the pump mechanism (e.g., pump mechanism 224) from a ratchet wheel directly (e.g., ratchet wheel 302a or 302b), a gear train may be actuated by the ratchet wheel to drive the pump mechanism.

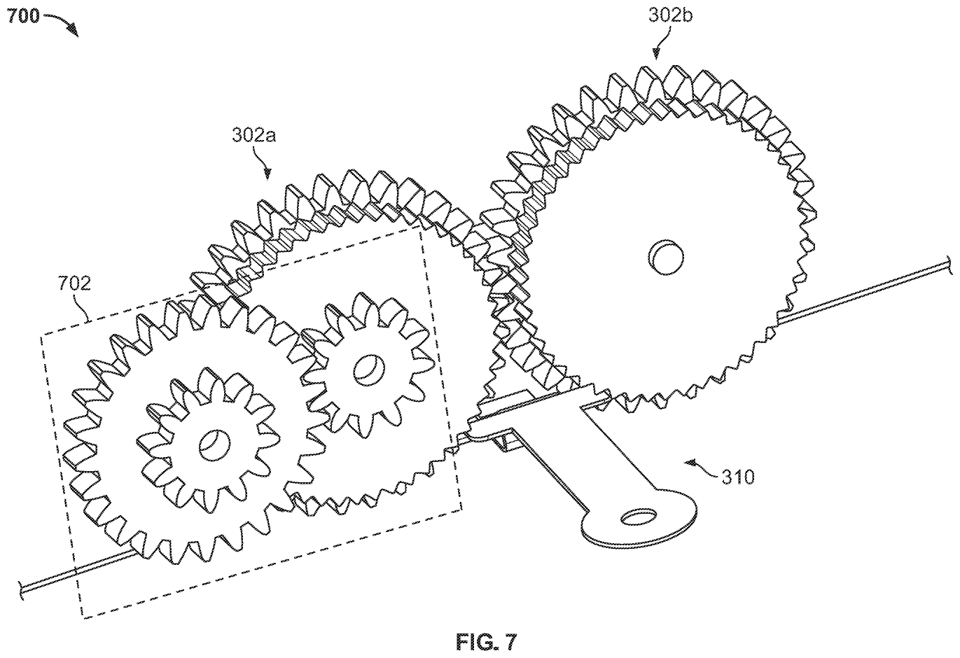

FIG. 7 illustrates a drive mechanism 700 in accordance with aspects described herein. In one example, the drive mechanism 700 is substantially the same as the drive mechanism 300 of FIGS. 3A-4B, except the drive mechanism 700 includes a gear train 702. As shown, the gear train 702 is coupled to the first ratchet wheel 302a and actuated via the rotation of the first ratchet wheel 302a provided by the actuation mechanism 310. In other examples, the gear train 702 can be coupled to the second ratchet wheel 302b and actuated via the rotation of the second ratchet wheel 302b. In some examples, the gear train 702 is configured as a set of interacting gears, a planetary gearbox, a harmonic gearbox, or any other suitable gear train configuration. The gear train 702 may include two or more gears that mesh (or otherwise interact) to provide a desired mechanical advantage. For example, the gear train 702 may be used to provide an increased mechanical advantage relative to the mechanical advantage provided directly by the ratchet wheels 302a, 302b. Two or more of the gears included in the gear train 702 may have different sizes (e.g., diameters). The reduction ratio of the gear train 702 may be tuned or adjusted to increase the input torque provided to the pump mechanism. In some examples, the gear train 702 may enable the pumping mechanism to operate with an increased dosing resolution (e.g., to deliver liquid drugs in smaller dose sizes). The gear train 702 may enable the dimensions (e.g., diameter, thickness, etc.) of the ratchet wheels 302a, 302b to be reduced. While not shown, the drive mechanism 700 may include a pawl mechanism (e.g., the pawl mechanism 602) operable to engage with the second ratchet wheel 302b to provide a stabilizing force to the ratchet wheels 302a, 302b and the gear train 702.

In the examples described above, the actuation mechanism 310 is positioned such that the pusher interface 312 is substantially perpendicular to the first ratchet wheel 302a. However, in other examples, the actuation mechanism may be positioned differently.

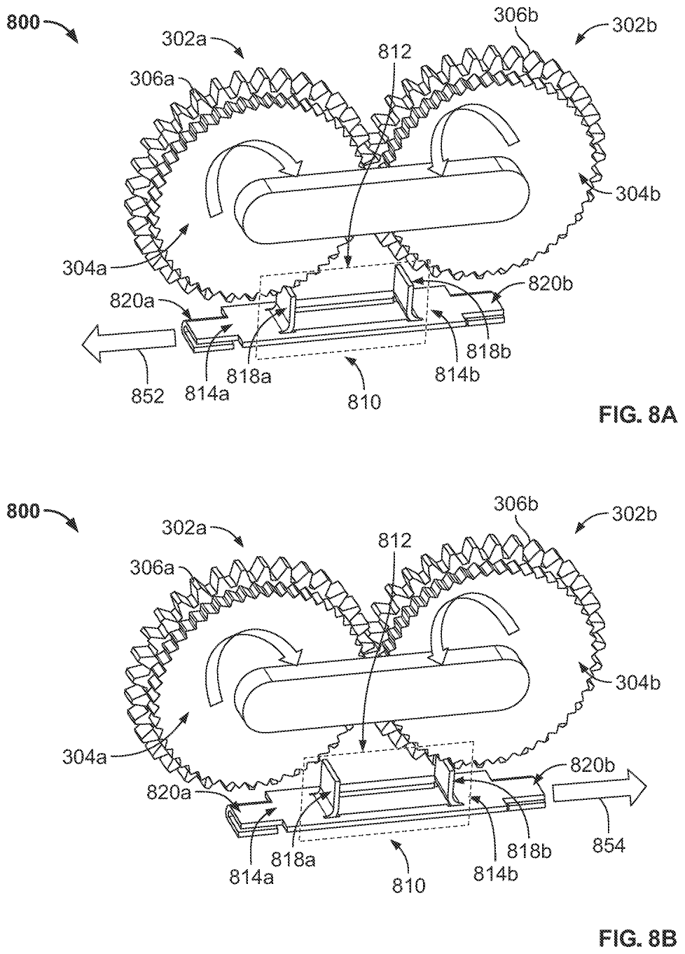

FIGS. 8A and 8B illustrate an example operation of a drive mechanism 800 of a liquid drug delivery device in accordance with aspects described herein. The drive mechanism 800 is substantially the same as the drive mechanism 300, except the drive mechanism 800 includes an actuation mechanism 810 positioned such that a pusher interface 812 is substantially parallel to the first ratchet wheel 302a (or the second ratchet wheel 302b). In one example, FIG. 8A represents a first operational state of the drive mechanism 800 and FIG. 8B represents a second operational state of the drive mechanism 800.

As shown in FIGS. 8A and 8B, the actuation mechanism 810 includes the pusher interface 812 and an actuator arm 814. In one example, the actuator arm 814 includes a first portion 814a and a second portion 814b. The pusher interface 812 is coupled between the first portion of the actuator arm 814a and the second portion of the actuator arm 814b. The pusher interface 812 includes a first pusher tab 818a and a second pusher tab 818b. The first pusher tab 818a is operable to physically contact the first plurality of ratchet gear teeth of the first ratchet gear 304a. In one example, the first pusher tab 818a extends substantially perpendicularly from a horizontal surface of the pusher interface 812 toward the first ratchet gear 304a. Likewise, the second pusher tab 818b is operable to physically contact the second plurality of ratchet gear teeth of the second ratchet gear 304b. In one example, the second pusher tab 818b extends substantially perpendicularly from a horizontal surface of the pusher interface 812 toward the second ratchet gear 304b. In some examples, the actuation mechanism 810 is positioned such that the pusher interface 812 is substantially parallel to the first ratchet wheel 302a. In other words, the pusher interface 812 may be substantially parallel to the gear face of the first ratchet wheel 302a (or the first ratchet gear 304a).

In some examples, the first portion of the actuator arm 814a is coupled to a first crimp connection 820a and the second portion of the actuator arm 814b is coupled to a second crimp connection 820b. In other examples, the crimp connections 820a, 820b can be coupled to the pusher interface 812. The crimp connections 820a, 820b are coupled to at least one actuator (not shown) via at least one biasing member (e.g., biasing members 322, 323 or biasing members 502a, 502b). In one example, the at least one biasing member causes the actuator arm 814 to swing along an axis substantially parallel to the first and second ratchet wheels 302a, 302b. The actuator arm 814 is operable to reciprocate to enable the pusher tabs 818a, 818b to contact the ratchet gears 304a, 304b. The at least one actuator may alternate between causing the first pusher tab 818a to contact the first plurality of ratchet gear teeth of the first ratchet gear 304a and causing the second pusher tab 818b to contact the second plurality of ratchet gear teeth of the second ratchet gear 304b. For example, the at least one actuator may pull the first crimp connection 820a via the at least one biasing member such that the first pusher tab 818a contacts a gear tooth of the first plurality of ratchet gear teeth. The first pusher tab 818a may be oriented to be parallel to a respective gear tooth (e.g., the gear tooth being contacted) of the first plurality of ratchet gear teeth of the first ratchet gear 304a. In some examples, the first pusher tab 818a is operable to contact an interior face and/or an exterior face of the first plurality of ratchet gear teeth of the first ratchet gear 304a as the actuator arm 814 reciprocates. Likewise, the at least one actuator may pull the second crimp connection 820b via the at least one biasing member such that the second pusher tab 818b contacts a gear tooth of the second plurality of ratchet gear teeth. The second pusher tab 818b may be oriented to be parallel to a respective gear tooth (e.g., the gear tooth being contacted) of the second plurality of ratchet gear teeth of the second ratchet gear 304b. In some examples, the second pusher tab 818b is operable to contact an interior face and/or an exterior face of the second plurality of ratchet gear teeth of the second ratchet gear 304b as the actuator arm 814 reciprocates.

As shown in FIG. 8A, in the first operational state, the actuation mechanism 810 is pulled via the first crimp connection 820a in a first linear direction 852. The actuation mechanism 810 slides in the first linear direction 852 causing the first pusher tab 818a to contact a respective gear tooth of the first plurality of ratchet gear teeth of the first ratchet gear 304a. The force applied to the respective gear tooth by the first pusher tab 818a causes the first ratchet gear 304a to rotate in a first rotational direction (e.g., clockwise). Being that the first ratchet gear 304a is coupled to the first spur gear 306a, the rotation of the first ratchet gear 304a causes the first spur gear 306a to rotate in the first rotational direction. The plurality of spur gear teeth of the first spur gear 306a apply a force to the plurality of spur gear teeth of the second spur gear 306b that causes the second spur gear 306b to rotate in a second rotational direction (e.g., counter-clockwise). Being that the second ratchet gear 304b is coupled to the second spur gear 306b, the rotation of the second spur gear 306b causes the second ratchet gear 304b to rotate in the second rotational direction. It should be appreciated that the second pusher tab 818b will be disengaged from the second ratchet gear 304b when the first pusher tab 818a is in contact with the first ratchet gear 304a (e.g., in the first operational state).

As shown in FIG. 8B, in the second operational state, the actuation mechanism 810 is pulled via the second crimp connection 820b in a second linear direction 854. The actuation mechanism 810 slides in the second linear direction 854 causing the second pusher tab 818b to contact a respective gear tooth of the second plurality of ratchet gear teeth of the second ratchet gear 304b. The force applied to the respective gear tooth by the second pusher tab 818b causes the second ratchet gear 304b to rotate in the second rotational direction. Being that the second ratchet gear 304b is coupled to the second spur gear 306b, the rotation of the second ratchet gear 304b causes the second spur gear 306b to rotate in the second rotational direction. The plurality of spur gear teeth of the second spur gear 306b apply a force to the plurality of spur gear teeth of the first spur gear 306a that causes the first spur gear 306a to rotate in the first rotational direction. Being that the first ratchet gear 304a is coupled to the first spur gear 306a, the rotation of the first spur gear 306a causes the first ratchet gear 304a to rotate in the first rotational direction. It should be appreciated that the first pusher tab 818a will be disengaged from the first ratchet gear 304a when the second pusher tab 818b is in contact with the second ratchet gear 304b (e.g., in the second state of operation).

In some examples, the positioning of the actuation mechanism relative to the ratchet wheels determines the rotational directions of the ratchet wheels. For example, in FIG. 3A, the actuation mechanism 310 is positioned at a lower portion of the ratchet wheels 302a, 302b (e.g., below the first and second shafts 308a, 308b). As such, the first pusher tab 318a causes the first ratchet wheel 302b to rotate clockwise and the second pusher tab 318b causes the second ratchet wheel 302b to rotate counter-clockwise. However, in other examples, the actuation mechanism can be positioned to provide different rotational arrangements.

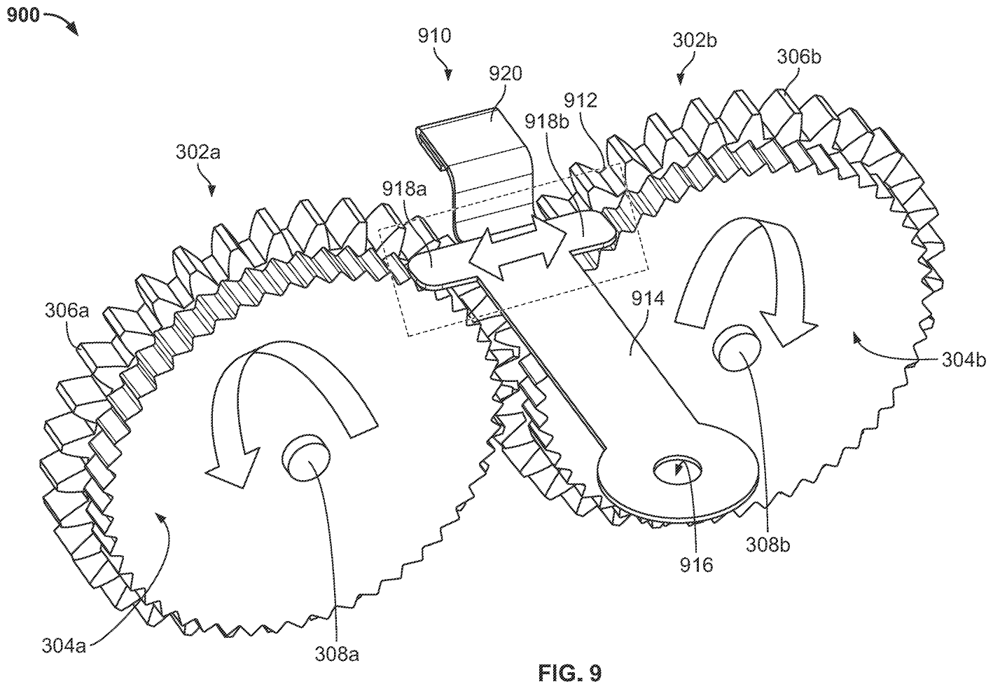

FIG. 9 illustrates a drive mechanism 900 of a liquid drug delivery device in accordance with aspects described herein. The drive mechanism 900 is substantially the same as the drive mechanism 300, except the drive mechanism 900 includes an actuation mechanism 910 positioned at a top portion of the ratchet wheels 302a, 302b (e.g., above the first and second shafts 308a, 308b). As such, the first ratchet wheel 302a rotates in the second rotational direction (e.g., counter-clockwise) and the second ratchet wheel 302b rotates in the first rotational direction (e.g., clockwise). In one example, the actuation mechanism 910 corresponds to the actuation mechanism 310 of the drive mechanism 300; however, in other examples, the actuation mechanism 1110 may correspond to the actuation mechanism 810 of the drive mechanism 800. In some examples, a configuration similar to the drive mechanism 900 can be achieved by rotating the drive mechanism 300 180 degrees.

In a first operational state, the actuation mechanism 910 may be pulled via the crimp connection 920 in a first linear direction. The actuator arm 914 rotates about the pivot point 916 causing the first pusher tab 918a to contact a respective gear tooth of the first plurality of ratchet gear teeth of the first ratchet gear 304a. The force applied to the respective gear tooth by the first pusher tab 918a causes the first ratchet gear 304a to rotate in the second rotational direction (e.g., counter-clockwise). Being that the first ratchet gear 304a is coupled to the first spur gear 306a, the rotation of the first ratchet gear 304a causes the first spur gear 306a to rotate in the second rotational direction. The plurality of spur gear teeth of the first spur gear 306a apply a force to the plurality of spur gear teeth of the second spur gear 306b that causes the second spur gear 306b to rotate in the first rotational direction (e.g., clockwise). Being that the second ratchet gear 304b is coupled to the second spur gear 306b, the rotation of the second spur gear 306b causes the second ratchet gear 304b to rotate in the first rotational direction.

In a second operational state, the actuation mechanism 910 may be pulled via the crimp connection 920 in a second linear direction. The actuator arm 914 rotates about the pivot point 916 causing the second pusher tab 918b to contact a respective gear tooth of the second plurality of ratchet gear teeth of the second ratchet gear 304b. The force applied to the respective gear tooth by the second pusher tab 918b causes the second ratchet gear 304b to rotate in the first rotational direction. Being that the second ratchet gear 304b is coupled to the second spur gear 306b, the rotation of the second ratchet gear 304b causes the second spur gear 306b to rotate in the first rotational direction. The plurality of spur gear teeth of the second spur gear 306b apply a force to the plurality of spur gear teeth of the first spur gear 306a that causes the first spur gear 306a to rotate in the second rotational direction. Being that the first ratchet gear 304a is coupled to the first spur gear 306a, the rotation of the first spur gear 306a causes the first ratchet gear 304a to rotate in the second rotational direction.

Alternating the drive mechanism 900 between the first and second operational states results in an incremental rotation of the ratchet wheels 302a, 302b. As such, by coupling one of the ratchet wheels 302a, 302b to a pumping system (e.g., pump mechanism 224), the incremental rotational motion can be used to actuate the pump and deliver a liquid drug to a patient.

It should be appreciated that any of the drive mechanisms described herein may include multiple linear actuation mechanisms. For example, a drive mechanism may include a first linear actuation mechanism positioned at the top portion of the ratchet wheels 302a, 302b (e.g., above the first and second shafts 308a, 308b) and a second linear actuation mechanism positioned at the bottom portion of the ratchet wheels 302a, 302b (e.g., below the first and second shafts 308a, 308b). In one example, the first and second linear actuation mechanisms each correspond to the actuation mechanism 310 of the drive mechanism 300. In other examples, the first linear actuation mechanism and/or the second linear actuation mechanism corresponds to the actuation mechanism 810 of the drive mechanism 800. Given that one of the ratchet wheels is coupled to the pumping system, the first and second linear actuation mechanisms may be controlled to operate the pumping system with bidirectional control. For example, the first linear actuation mechanism may be used to rotate the first ratchet wheel in the first rotational direction (e.g., clockwise) and the second ratchet wheel in the second rotational direction (e.g., counter-clockwise). The rotation of the first ratchet wheel or the second ratchet wheel may cause the pump to dispense the liquid drug from the reservoir (e.g., by driving a plunger forward). Likewise, the second linear actuation mechanism may be used to rotate the first ratchet wheel in the second rotational direction and the second ratchet wheel in the first rotational direction. Reversing the rotation of the first ratchet wheel or the second ratchet wheel may cause the pump to return to an original state (e.g., by driving the plunger in reverse). As such, the second linear actuation mechanism may enable the reservoir of the pumping system to be re-filled for multiple uses.

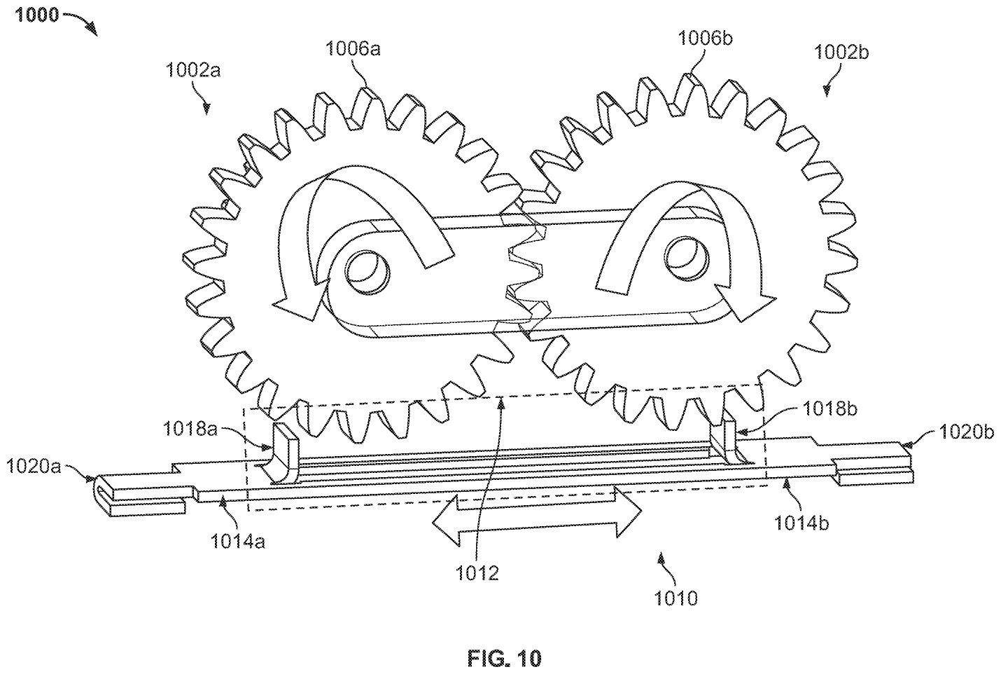

While the drive mechanisms described above include linear actuation mechanisms that engage with the ratchet gear teeth of the ratchet gear wheels, in some examples the ratchet gear wheels may be optional. For example, FIG. 10 illustrates a drive mechanism 1000 of a liquid drug delivery device in accordance with aspects described herein. The drive mechanism 1000 includes a first ratchet wheel 1002a, a second ratchet wheel 1002b, and an actuation mechanism 1010. In one example, the first ratchet wheel 1002a includes a first spur gear 1006a and the second ratchet wheel 1002b includes a second spur gear 1006b. In some examples, the actuation mechanism 1010 is substantially the same as the actuation mechanism 810 of the drive mechanism 800 of FIG. 8, except the actuation mechanism 1010 is configured to contact the spur gear teeth of the first and second spur gears 1006a, 1006b.

As shown, the actuation mechanism 1010 includes a pusher interface 1012 and an actuator arm 1014. In one example, the actuator arm 1014 includes a first portion 1014a and a second portion 1014b. The pusher interface 1012 is coupled between the first portion of the actuator arm 1014a and the second portion of the actuator arm 1014b. The pusher interface 1012 includes a first pusher tab 1018a and a second pusher tab 1018b. In one example, the first pusher tab 1018a is operable to physically contact the first plurality of spur gear teeth of the first spur gear 1006a and the second pusher tab 1018b is operable to physically contact the second plurality of spur gear teeth of the second spur gear 1006b. In some examples, the actuation mechanism 1010 is positioned such that the pusher interface 1012 is substantially parallel to the first spur gear 1006a. In other words, the pusher interface 1012 may be substantially parallel to a gear face of the first spur gear 1006a.

In some examples, the first portion of the actuator arm 1014a is coupled to a first crimp connection 1020a and the second portion of the actuator arm 1014b is coupled to a second crimp connection 1020b. In other examples, the crimp connections 1020a, 1020b can be coupled to the pusher interface 1012. The crimp connections 1020a, 1020b are coupled to at least one actuator (not shown) via at least one biasing member (e.g., biasing member 322 and/or 323 or biasing members 502a, 502b). The at least one actuator may alternate between causing the first pusher tab 1018a to contact the first plurality of spur gear teeth of the first spur gear 1006a and causing the second pusher tab 1018b to contact the second plurality of spur gear teeth of the second spur gear 1006b. For example, the at least one actuator may pull the first crimp connection 1020a via the at least one biasing member such that the first pusher tab 1018a contacts a gear tooth of the first plurality of spur gear teeth. The first pusher tab 1018a may be oriented to be parallel to a respective gear tooth (e.g., the gear tooth being contacted) of the plurality of spur gear teeth of the first spur gear 1006a. Likewise, the at least one actuator may pull the second crimp connection 1020b via the at least one biasing member such that the second pusher tab 1018b contacts a gear tooth of the second plurality of spur gear teeth. The second pusher tab 1018b may be oriented to be parallel to a respective gear tooth (e.g., the gear tooth being contacted) of the plurality of spur gear teeth of the second spur gear 1006b.

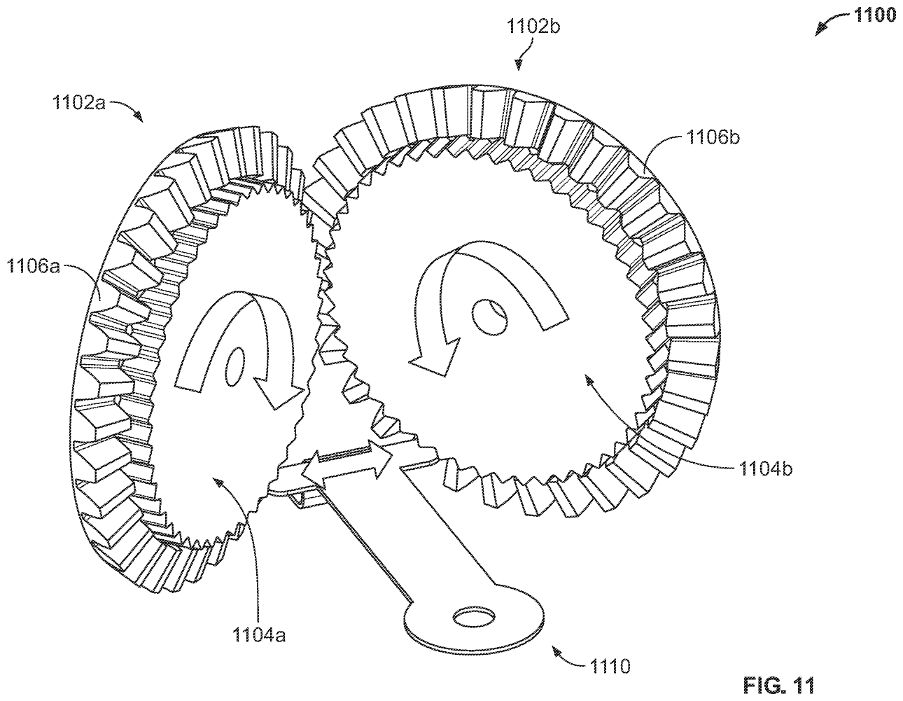

Depending on the location of the drive mechanism within the liquid drug delivery device, it may be advantageous to use a drive mechanism including non-planar ratchet wheels. For example, FIG. 11 illustrates a drive mechanism 1100 including non-planar ratchet wheels 1102a, 1102b in accordance with aspects described herein. As shown, the angle between the ratchet wheels 1102a, 1102b is greater than 0 degrees and less than 180 degrees. In one example, the first ratchet wheel 1102a includes a first ratchet gear 1104a and a first spur gear 1106a and the second ratchet wheel 1102b includes a second ratchet gear 1104b and a second spur gear 1106a. In some examples, the first and second spur gears 1106a, 1106b are configured as bevel gears. For example, the first spur gear 1106a is beveled and the second spur gear 1106b is beveled to mesh with the beveled first spur gear 1106a. The drive mechanism 1100 may operate similarly to the drive mechanism 300 in that the actuation mechanism 1110 is actuated to engage with the gear teeth of the first ratchet gear 1104a (or the first spur gear 1106a) and the second ratchet gear 1104b (or the second spur gear 1106b) to rotate the ratchet wheels 1102a, 1102b.

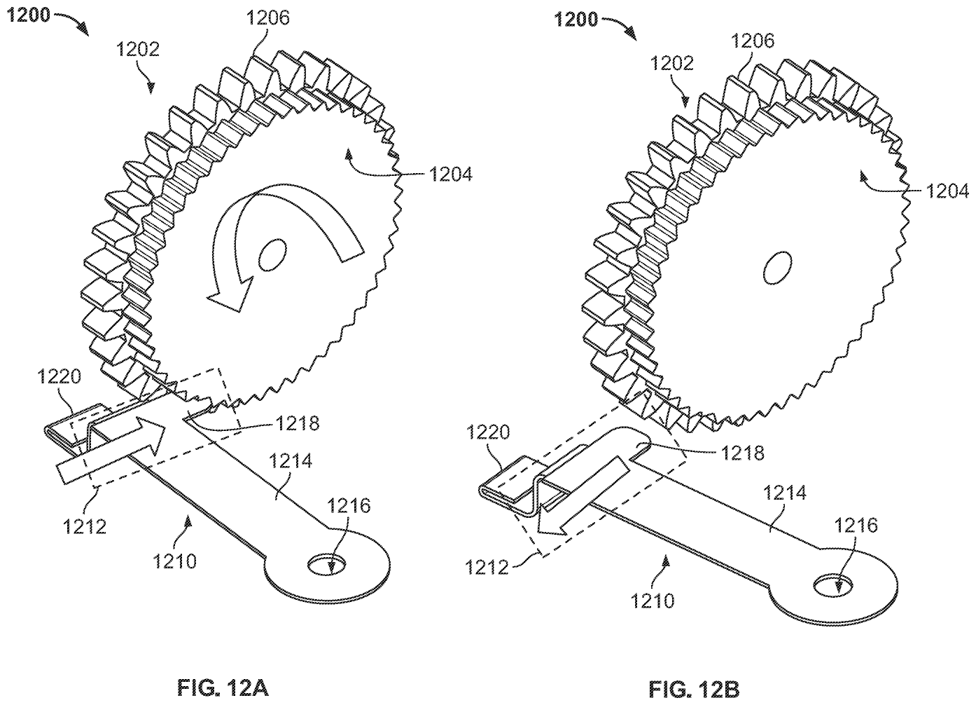

Depending on the configuration of the pump mechanism and/or the liquid drug delivery device, it may be advantageous to use a drive mechanism including a single ratchet wheel. For example, FIGS. 12A and 12B illustrate a drive mechanism 1200 including a ratchet wheel 1202 in accordance with aspects described herein. The drive mechanism 1200 includes an actuation mechanism 1210 having a pusher interface 1212 with a single pusher tab 1218. In one example, FIG. 12A represents a first operational state of operation of the drive mechanism 1200 and FIG. 12B represents a second operational state of operation of the drive mechanism 1200.

As shown in FIG. 12A, in the first operational state, the actuation mechanism 1210 is pulled via the crimp connection 1220 in a first linear direction. In one example, the crimp connection 1220 is coupled to at least one biasing member (e.g., biasing member 322 and/or 323 or biasing members 502a, 502b). The actuator arm 1214 rotates about the pivot point 1216 causing the pusher tab 1218 to contact a respective gear tooth of the first plurality of ratchet gear teeth of the ratchet gear 1204. The force applied to the respective gear tooth by the pusher tab 1218 causes the ratchet gear 1204 to rotate in a rotational direction (e.g., counter-clockwise). Being that the ratchet gear 1204 is coupled to the spur gear 1206, the rotation of the ratchet gear 1204 causes the spur gear 1206 to rotate in the same rotational direction.

As shown in FIG. 12B, in the second operational state, the actuation mechanism 1210 is pulled via the crimp connection 1220 in a second linear direction. The actuator arm 1214 rotates about the pivot point 1216 causing the second pusher tab 1218 to disengage from the ratchet gear teeth of the ratchet gear 1204. As such, the ratchet wheel 1202 (e.g., the ratchet gear 1204 and the spur gear 1206) may be stationary in the second operational state. In some examples, the drive mechanism 1200 includes at least one pawl mechanism (e.g., pawl mechanism 602) configured to stabilize the ratchet wheel 1202 during the second operational state.

Alternating the drive mechanism 1200 between the first and second operational states results in an incremental rotation of the ratchet wheel 1202. As such, by coupling the ratchet wheel 1202 to a pumping system (e.g., pump mechanism 224), the incremental rotational motion can be used to actuate the pump and deliver a liquid drug to a patient. It should be appreciated that the spur gear 1206 may be optional.

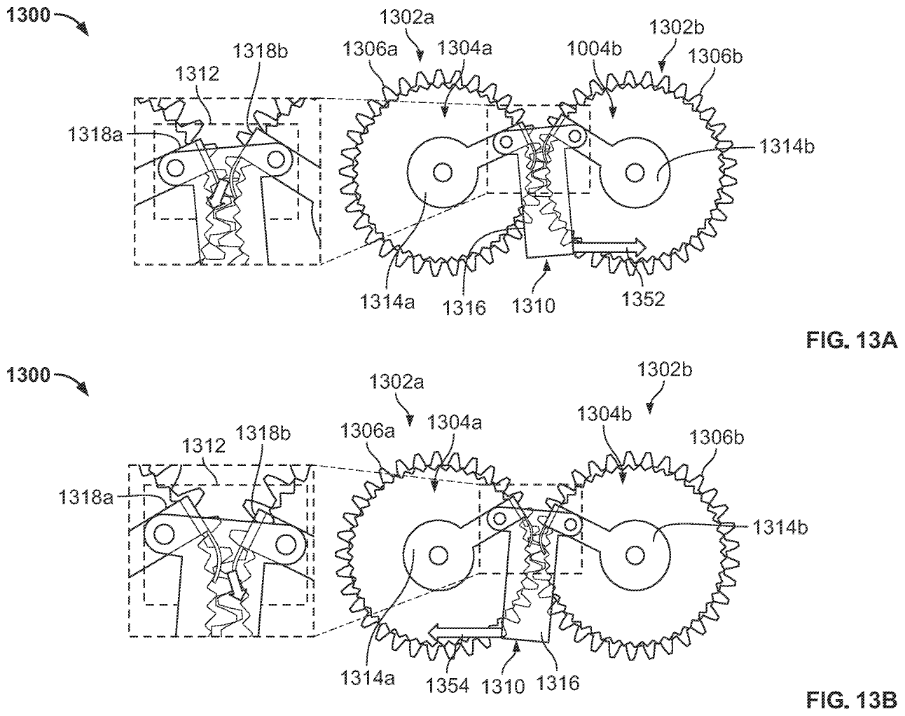

FIGS. 13A and 13B illustrate an example operation of an alternative drive mechanism 1300 of a liquid drug delivery device in accordance with aspects described herein. The drive mechanism 1300 includes an actuation mechanism 1310 that is substantially in-plane with the ratchet wheels 1302a, 1302b. In one example, FIG. 13A represents a first operational state of the drive mechanism 1300 and FIG. 13B represents a second operational state of the drive mechanism 1300.

The actuation mechanism 1310 includes a pusher interface 1312, a linkage mechanism 1314, and an actuator arm 1316. In one example, the linkage mechanism 1314 includes a first portion 1314a coupled to the first ratchet wheel 1302a (e.g., to the first ratchet gear 1304a) and a second portion 1314b coupled to the second ratchet wheel 1302b (e.g., to the second ratchet gear 1304b).

The pusher interface 1312 is coupled between the first portion of the linkage mechanism 1314a, the second portion of the linkage mechanism 1314b, and the actuator arm 1316. The pusher interface 1312 includes a first pusher tab 1318a and a second pusher tab 1318b. In one example, the first pusher tab 1318a is operable to physically contact the first plurality of ratchet gear teeth of the first ratchet gear 1304a and the second pusher tab 1318b is operable to physically contact the second plurality of ratchet gear teeth of the second ratchet gear 1304b. In some examples, the actuation mechanism 1310 is positioned such that the pusher interface 1312 is substantially in-plane with the first ratchet wheel 1302a. In other words, the pusher interface 1312 may be substantially co-planar with the gear face of the first ratchet wheel 1302a (or the first ratchet gear 1304a).

In some examples, the actuator arm 1310 is coupled to a crimp connection (e.g., crimp connection 320). The crimp connection is coupled to at least one actuator (not shown) via at least one biasing member (e.g., biasing member 322 and/or 323 or biasing members 502a, 502b). The at least one actuator may alternate between causing the first pusher tab 1318a to contact the first plurality of ratchet gear teeth of the first ratchet gear 1304a and causing the second pusher tab 1318b to contact the second plurality of ratchet gear teeth of the second ratchet gear 1304b. For example, the at least one actuator may pull the crimp connection via the at least one biasing member such that the first pusher tab 1318a contacts the first plurality of ratchet gear teeth. Likewise, the at least one actuator may pull the crimp connection via the at least one biasing member such that the second pusher tab 1318b contacts the second plurality of ratchet gear teeth.

As shown in FIG. 13A, in the first operational state, the actuation mechanism 1310 is pulled via the crimp connection in a first linear direction 1352. The actuation mechanism 1310 pivots in the first linear direction 1352 causing the first pusher tab 1318a to contact a respective gear tooth of the first plurality of ratchet gear teeth of the first ratchet gear 1304a. The force applied to the respective gear tooth by the first pusher tab 1318a causes the first ratchet gear 1304a to rotate in the first rotational direction (e.g., clockwise). Being that the first ratchet gear 1304a is coupled to the first spur gear 1306a, the rotation of the first ratchet gear 1304a causes the first spur gear 1306a to rotate in the first rotational direction. The plurality of spur gear teeth of the first spur gear 1306a apply a force to the plurality of spur gear teeth of the second spur gear 1306b that causes the second spur gear 1306b to rotate in the second rotational direction (e.g., counter-clockwise). Being that the second ratchet gear 1304b is coupled to the second spur gear 1306b, the rotation of the second spur gear 1306b causes the second ratchet gear 1306b to rotate in the second rotational direction. It should be appreciated that the second pusher tab 1318b will be disengaged from the second ratchet gear 1304b when the first pusher tab 1318a is in contact with the first ratchet gear 1304a (e.g., in the first operational state).

As shown in FIG. 13B, in the second operational state, the actuation mechanism 1310 is pulled via the crimp connection in a second linear direction 1354. The actuation mechanism 1310 pivots in the second linear direction 1354 causing the second pusher tab 1318b to contact a respective gear tooth of the second plurality of ratchet gear teeth of the second ratchet gear 1304b. The force applied to the respective gear tooth by the second pusher tab 1318b causes the second ratchet gear 1304b to rotate in the second rotational direction. Being that the second ratchet gear 1304b is coupled to the second spur gear 1306b, the rotation of the second ratchet gear 1304b causes the second spur gear 1306b to rotate in the second rotational direction. The plurality of spur gear teeth of the second spur gear 1306b apply a force to the plurality of spur gear teeth of the first spur gear 1306a that causes the first spur gear 1306a to rotate in the first rotational direction. Being that the first ratchet gear 1304a is coupled to the first spur gear 1306a, the rotation of the first spur gear 1306a causes the first ratchet gear 1306a to rotate in the first rotational direction. It should be appreciated that the first pusher tab 1318a will be disengaged from the first ratchet gear 1304a when the second pusher tab 1318b is in contact with the second ratchet gear 1304b (e.g., in the second operational state).

As described above, improved drug delivery device drive mechanisms are provided herein. In at least one embodiment, the drive mechanism is a dual wheel actuator with coplanar ratchet wheels. In one example, a first ratchet wheel includes a first spur gear having gear teeth that mesh with gear teeth of a second spur gear of a second ratchet wheel. In some examples, the co-planar arrangement of the first and second ratchet wheels allows the drive mechanism to have a compact form factor.

The techniques described herein for a drug delivery system (e.g., the system 100, the system 200, or any components thereof) may be implemented in hardware, software, or any combination thereof. Any component as described herein may be implemented in hardware, software, or any combination thereof. For example, the systems 100, 200 or any components thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Some examples of the disclosed devices may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

As used herein, the algorithms or computer applications that manage blood glucose levels and insulin therapy may be referred to as an “artificial pancreas” algorithm-based system, or more generally, an artificial pancreas (AP) application. An AP application may be programming code stored in a memory device and that is executable by a processor, controller or computer device.

Certain examples of the present disclosed subject matter were described above. It is, however, expressly noted that the present disclosed subject matter is not limited to those examples, but rather the intention is that additions and modifications to what was expressly described herein are also included within the scope of the disclosed subject matter. Moreover, it is to be understood that the features of the various examples described herein were not mutually exclusive and may exist in various combinations and permutations, even if such combinations or permutations were not made express herein, without departing from the spirit and scope of the disclosed subject matter. In fact, variations, modifications, and other implementations of what was described herein will occur to those of ordinary skill in the art without departing from the spirit and the scope of the disclosed subject matter. As such, the disclosed subject matter is not to be defined only by the preceding illustrative description.

In particular, although the previous embodiments have been described in context with first and second ratchet gears, it should be understood that the actuation mechanism may also engage and drive the first and second spur gears. In this case, first and second ratchet gears are not needed, i.e. he spur gears have a dual function, namely to mesh with each other to achieve a coupled rotation (in opposite directions) and to cooperate with the at least one actuator which drives the first spur gear and/or the second spur gear.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of example examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A drug delivery device drive mechanism, comprising:
a first ratchet wheel having a first ratchet gear coupled to a first spur gear;
a second ratchet wheel having a second ratchet gear coupled to a second spur gear, wherein the first ratchet wheel and the second ratchet wheel are coplanar and gear teeth of the first spur gear mesh with gear teeth the second spur gear; and
a linear actuation mechanism including a pusher interface coupled to an actuator, the pusher interface having at least one pusher tab operable to physically contact the first ratchet gear.

2. The drug delivery device drive mechanism of claim 1, wherein the pusher interface is substantially perpendicular to a gear face of the first ratchet wheel.

3. The drug delivery device drive mechanism of claim 1, wherein the pusher interface is substantially parallel to a gear face of the first ratchet wheel.

4. The drug delivery device drive mechanism of claim 1, wherein the at least one pusher tab of the pusher interface further comprises:
a first pusher tab operable to contact a respective gear tooth of a plurality of gear teeth of the first spur gear; and
a second pusher tab operable to contact a respective gear tooth of a plurality of gear teeth of the second spur gear,
wherein the actuator alternates between causing the first pusher tab to contact the respective gear tooth of the plurality of gear teeth of the first spur gear and causing the second pusher tab to contact the respective gear tooth of the plurality of gear teeth of the second spur gear.

5. The drug delivery device drive mechanism of claim 4, wherein the first ratchet wheel rotates about a first axis in a first direction and the second ratchet wheel rotates about a second axis in a second direction.

6. The drug delivery device drive mechanism of claim 5, wherein the second axis is parallel to the first axis.

7. The drug delivery device drive mechanism of claim 5, wherein the first direction is opposite the second direction.

8. The drug delivery device drive mechanism of claim 5, wherein the first pusher tab contacts the respective gear tooth of the plurality of gear teeth of the first ratchet gear to rotate the first ratchet wheel about the first axis in the first direction causing the second ratchet wheel to rotate about the second axis in the second direction.

9. The drug delivery device drive mechanism of claim 5, wherein the second pusher tab contacts the respective gear tooth of the plurality of gear teeth of the second ratchet gear to rotate the second ratchet wheel about the second axis in the second direction causing the first ratchet wheel to rotate about the first axis in the first direction.

10. The drug delivery device drive mechanism of claim 4, wherein:
the first pusher tab is oriented to be perpendicular to the respective gear tooth of the plurality of gear teeth of the first ratchet gear, and
the second pusher tab is oriented to be perpendicular to the respective gear tooth of the plurality of gear teeth of the second ratchet gear.

11. The drug delivery device drive mechanism of claim 4, wherein:
the first pusher tab is oriented to be parallel to the respective gear tooth of the plurality of gear teeth of the first ratchet gear, and
the second pusher tab is oriented to be parallel to the respective gear tooth of the plurality of gear teeth of the second ratchet gear.

12. The drug delivery device drive mechanism of claim 1, further comprising:
a pawl having an edge that rests between gear teeth of either the first spur gear or the second spur gear, wherein the pawl is operable to prevent free rotation of either the first ratchet wheel or the second ratchet wheel.

13. The drug delivery device drive mechanism of claim 1, wherein the linear actuation mechanism further comprises:
an actuator arm coupled to the pusher interface, the actuator arm including a first end coupled to a pivot point and a second end coupled to the pusher interface, wherein the first and second ends are opposite ends of the actuator arm.

14. The drug delivery device drive mechanism of claim 13, wherein the pusher interface comprises:
a crimp connection operable to couple to a shape-memory alloy wire, and operation of the shape-memory alloy wire causes the actuator arm to swing with respect to the pivot point.

15. The drug delivery device drive mechanism of claim 1, wherein the linear actuation mechanism further comprises:
a linear actuator arm, wherein the pusher interface is coupled to the linear actuator arm and the at least one pusher tab extends substantially perpendicularly from a horizontal surface of the pusher interface toward the first ratchet gear, and the linear actuator arm is operable to reciprocate to enable the at least one pusher tab to contact the first ratchet gear, and
an actuator connection point at which the actuator is coupled to an end of the linear actuator arm.

16. The drug delivery device drive mechanism of claim 15, wherein the gear teeth of the first ratchet gear and the gear teeth the second ratchet gear have an interior face and an exterior face, and the at least one pusher tab is operable to contact the interior face of the first ratchet gear and the second ratchet gear as the linear actuator arm reciprocates.

17. The drug delivery device drive mechanism of claim 15, wherein the gear teeth of the first ratchet gear have an interior face and an exterior face and the gear teeth the second ratchet gear have an interior face and an exterior face, and the at least one pusher tab is operable to contact the exterior face of the first ratchet gear and the second ratchet gear as the linear actuator arm reciprocates.

18. The drug delivery device drive mechanism of claim 15, wherein the actuator connection point comprises:
a crimp connection operable to couple to a shape-memory alloy wire, and operation of the shape-memory alloy wire causes the linear actuator arm to swing along an axis substantially parallel to the first ratchet wheel and the second ratchet wheel.

19. The drug delivery device drive mechanism of claim 15, wherein the actuator connection point comprises:
a first crimp connection operable to couple to a shape-memory allow wire and a second crimp connection operable to couple to a biasing member.

20. The drug delivery device drive mechanism of claim 1, further comprising:
a pump mechanism coupling operable to connect either the first ratchet wheel or the second ratchet wheel to a drive element.
